(19) United States
(12) Patent Application Publication
(10) Pub. No.: US 2023/0230684 A1
(43) Pub. Date: Jul. 20, 2023

(54) CATHETER DESIGN TOOL AND METHODS THEREOF

(71) Applicant: Medtronic, Inc., Minneapolis, MN (US)

(72) Inventors: Megan Samec, Minneapolis, MN (US); Varun A. Bhatia, Minneapolis, MN (US); Ryan P. Lahm, Lino Lakes, MN (US); Walton W. Baxter, San Clemente, CA (US); Brian P. Colin, Anoka, MN (US); Jay T. Rassat, Otsego, MN (US); Jeffrey G. Falk, Ham Lake, MN (US); Keith D. Anderson, Minneapolis, MN (US)

(21) Appl. No.: 18/083,283

(22) Filed: Dec. 16, 2022

Related U.S. Application Data
(60) Provisional application No. 63/299,505, filed on Jan. 14, 2022.

(51) Int. Cl. G16H 40/20; G16H 10/60; G16H 20/40; G16H 30/40

(52) U.S. Cl. CPC G16H 40/20; G16H 10/60; G16H 20/40; G16H 30/40

(57) ABSTRACT

The present disclosure provides a catheter design tool and methods thereof for selecting or designing an optimal catheter profile for a particular patient or group of patients. For example, the tool includes a data set (e.g., input into the system) pertaining to a plurality of anatomy parameters and a plurality of catheter parameters to determine one or more catheter profiles that allow the best implant efficacy for a particular patient or group of patients. Specifically, the tool evaluates the efficacy of a particular catheter profile for use with a particular patient, the most optimal catheter profile from a plurality of catheter profiles for use with a particular patient, and/or the most optimal catheter profile from a plurality of catheter profiles for use with a group of patients.

The present application claims the benefit of U.S. Provisional Application No. 63/299,505, filed Jan. 14, 2022, which is incorporated herein by reference in its entirety.

The disclosure generally relates to medical devices and, in particular, selecting and designing catheter profiles based on a plurality of anatomy parameters that govern how well a user can deliver therapy (e.g., drug, device, biologic, etc.) to a target in a patient or animal.

Often catheter design is incredibly slow and expensive due to the design-build-test-implant cycle that can require a lot of time and expense. Additionally, it may be more challenging for a catheter to reach a specific anatomic target when used on a broad population of patients (e.g., especially when used with pediatric patients). Further, predicting which catheter may be utilized for a particular patient may prove to be difficult due to the various existing characteristics of already developed catheters (e.g., having varying shapes and stiffness distributions) such that a user may rely on their own preference of catheter over which catheter may best suit the anatomy of the patient. Therefore, it may be desirable to have a tool that can guide the design of a catheter that is most suitable for a group of individuals based on a particular class of anatomies and anatomic targets. Further, it may be desirable to have a tool that can provide preoperative guidance showing an overlay of medical images and a predicted catheter path. Additionally, it may be desirable to have a tool to easily determine the most suitable catheter for an individual or group of individuals.

### SUMMARY

The techniques of the present disclosure generally relate to a tool that can be used to select and/or design one or more catheter profiles for a particular individual or group of individuals. For example, catheter profiles may be designed to quickly serve a patient population of interest by incorporating realistic anatomic measurements, enforcing heuristics for a particular catheter design (e.g., stiff sections for a first length, compliant sections for a second length, etc.), and providing data regarding successful catheter paths within an anatomy. Also, for example, the utility of an existing catheter design may be estimated in a new population based on provided anatomic data from that population. Specifically, an existing catheter profile may be selected for a particular patient or patient population using, for example, preoperative imaging or anatomic correlation with a specific demographic (e.g., patient height, body mass index (BMI), weight, ethnicity, gender, disease state, etc.).

The tool may operate in a variety of different ways to select or design one or more optimal catheter profiles for a patient or group of patients. For example, the tool may gather a data set including, e.g., anatomy/catheter design bounds, compute valid catheter paths, and obtain new/optimized catheter shape parameters that would drive implant success in a particular population (e.g., having those anatomy design bounds). Specifically, in application, the tool may include preoperative/perioperative decision making, backwards-looking judgments about how current catheter profiles have performed in the past, intraoperative guidance, and optimizing future catheter designs. In one or more embodiments, the tool may include determining one or more optimal catheter profiles from a plurality of catheter profiles based on a plurality of anatomy parameters and a plurality of catheter parameters. Also, in one or more embodiments, the tool may include evaluating a catheter profile in view of a plurality of anatomy parameters and a plurality of catheter parameters. Further, in one or more embodiments, the tool may include determining guidance for navigating a catheter profile within an anatomy based on a plurality of anatomy parameters and a plurality of catheter parameters. Further yet, in one or more embodiments, the tool may include designing one or more optimal catheter profiles based on a catheter path having a plurality of anatomy parameters.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a flow diagram that illustrates one example of a method for determining one or more optimal catheter profiles from a plurality of catheter profiles according to the present disclosure.

FIG. 2 is a conceptual diagram of a plurality of catheter profiles positioned with an anatomy along a variety of different paths according to the present disclosure.

FIG. 3 is a flow diagram that illustrates one example of a method for evaluating a catheter profile in view of a plurality of anatomy parameters according to the present disclosure.

FIG. 4A is a representative image of an illustrative human heart having one configuration of an atrial superior vena cava (SVC)/inferior vena cava (IVC) junction.

FIG. 4B is a representative image of another illustrative human heart having a different configuration of the atrial superior vena cava (SVC)/inferior vena cava (IVC) junction as compared to FIG. 4A.

FIG. 4C is a representative image of tool design (e.g., catheter profile) for a human implant shown in relation to an animal heart anatomy.

FIG. 5 is a flow diagram that illustrates one example of a method for determining guidance for navigating a catheter profile within an anatomy according to the present disclosure.

FIG. 6 is a flow diagram that illustrates one example of a method for designing one or more optimal catheter profiles having a plurality of catheter parameters according to the present disclosure.

FIG. 7 is a schematic illustration of a system including an exemplary system for selecting or designing an optimal catheter profile for a particular patient or group of patients.

### DETAILED DESCRIPTION

The present disclosure generally provides a catheter design tool or system and methods thereof for selecting or designing an optimal catheter profile for a particular patient or group of patients. For example, the tool or system may include a data set (e.g., input into the system) pertaining to a plurality of anatomy parameters and a plurality of catheter parameters to determine one or more catheter profiles that allow the best implant efficacy for a particular patient or group of patients. Specifically, the tool may evaluate the efficacy of a particular catheter profile for use with a particular patient, the most optimal catheter profile from a plurality of catheter profiles for use with a particular patient, and/or the most optimal catheter profile from a plurality of catheter profiles for use with a group of patients (e.g., divided by anatomic measurements or anatomic correlation with demographics). In other words, the tool may increase efficiency by selecting or designing the most effective catheter profile for a particular anatomy and eliminating potential waste created by using multiple catheter profiles to find the “best” catheter profile. It is noted that the term “catheter profile” as described herein may include a physical catheter (and the features thereof) or a representation of a catheter (and the features thereof).

Additionally, the data set (particularly the plurality of anatomy parameters) may be used to determine an optimal catheter path within one or more anatomies such that an optimal catheter profile may be designed having a plurality of catheter parameters. For example, the optimal catheter profile may be designed to be suitable for a particular anatomy or for a majority of anatomies (e.g., within a group). Furthermore, the tool may be used to provide guidance for navigating a catheter profile within an anatomy based on the data set.

As used herein, the term “or” refers to an inclusive definition, for example, to mean “and/or” unless its context of usage clearly dictates otherwise. The term “and/or” refers to one or all of the listed elements or a combination of at least two of the listed elements.

As used herein, the phrases “at least one of” and “one or more of” followed by a list of elements refers to one or more of any of the elements listed or any combination of one or more of the elements listed.

All scientific and technical terms used herein have meanings commonly used in the art unless otherwise specified. The definitions provided herein are to facilitate understanding of certain terms used frequently herein and are not meant to limit the scope of the present disclosure.

Reference will now be made to the drawings, which depict one or more aspects described in this disclosure. However, it will be understood that other aspects not depicted in the drawings fall within the scope of this disclosure. Like numbers used in the figures refer to like components, steps, and the like. However, it will be understood that the use of a reference character to refer to an element in a given figure is not intended to limit the element in another figure labeled with the same reference character. In addition, the use of different reference characters to refer to elements in different figures is not intended to indicate that the differently referenced elements cannot be the same or similar.

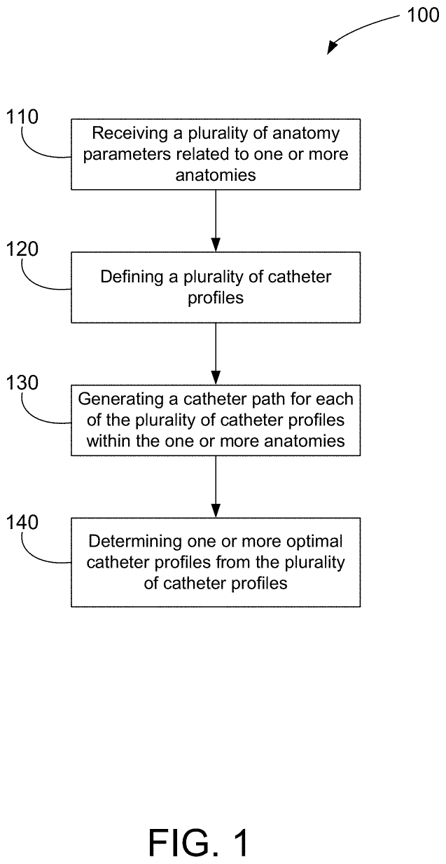

FIG. 1 shows a process 100 of determining one or more optimal catheter profiles from a plurality of catheter profiles based on a plurality of catheter parameters of the plurality of catheter profiles and a plurality of anatomy parameters. For example, there may be various instances in which a most optimal catheter profile is selected for an individual person (e.g., having a specific anatomy) or a group of people (e.g., having various anatomies).

Specifically, this information may be useful in settings including, e.g., in a clinic or operating room when treating a single patient, when determining which catheter profile functions most optimally for a specific group of people, when presented with a collection of various catheter profiles and determining which functions most optimally, when ranking a collection of various catheter profiles for one person or multiple people, etc.

The process 100 may include receiving 110 a plurality of anatomy parameters related to one or more anatomies. In other words, specific parameters or characteristics of a patient's anatomy may be input into a tool (e.g., through a system or controller) in order to evaluate the catheter profile. The plurality of anatomy parameters may include venous or arterial boundary shapes and distances, locations of anatomic targets relative to other anatomic landmarks, atrial wall locations, valve structure, coronary sinus ostia, coronary artery takeoffs, ventricular free and septal wall locations, orientation, endocardial shape (e.g., concave, convex), etc.

The plurality of anatomy parameters may be derived in a variety of different ways. For example, the one or more anatomies may be imaged to determine the plurality of anatomy parameters. Specifically, the imaging may include magnetic resonance imaging (MRI), fluoroscopy, cineradiography, biplane fluoroscopy, biplane cineradiography, computed tomography (CT), intraoperative 2D/3D imaging system (O-Arm), ultrasound (US), a transesophageal echocardiography (TEE), intra-cardiac echocardiography (ICE), transthoracic echocardiography (TTE), intravascular ultrasound (IVUS), electromechanical wave imaging (EWI), neuro-endoscopy, single photon emission computed tomography (SPECT), magnetic resonance angiography (MRA), computed tomography angiography (CTA), positron emission tomography (PET), optical coherence tomography (OCT), optical imaging spectroscopy (OIS), a magnetic resonance spectroscopy (MRS), dynamic susceptibility contrast (DSC) MRI, a fluid-attenuated inversion recovery (FLAIR), rotational angiography, infrared (IR) imaging, ultraviolet (UV) imaging, and/or the like. Also, the one or more anatomies may be measured in any suitable way such as, e.g., by segmenting anatomic boundaries from volumetric images (e.g., to extract anatomic boundaries) or by computing a best-fit volume from two or more perspective images of an anatomy. In one or more embodiments, 2D images (e.g., ultrasound planes) of the one or more anatomies may be measured by identifying (e.g., either manually or automatically) landmarks important to the performance of the catheter profile. Further, a user may manually input the plurality of anatomy parameters into a system or controller of the tool (e.g., due to known values of the plurality of anatomy parameters).

Additionally, in one or more embodiments, the plurality of anatomy parameters may be provided through a database of biometric information. The plurality of anatomy parameters may be selected and input as desired by a user to represent the one or more anatomies. For example, the one or more anatomies may correlate to a demographic group. Further, in one or more embodiments, the one or more anatomies may be grouped into a plurality of demographic groups. Specifically, the demographic group may be identified within one or more of, e.g., height, body mass index (BMI), ethnicity, gender, disease state, etc. In other words, there may be some correlation between one or more anatomy parameters of the plurality of anatomy parameters when the one or more anatomies are grouped by demographic group. Therefore, the demographic group may be used to approximate one or more anatomy parameters to determine one or more optimal catheter profiles for the anatomies of the demographic group, as will be discussed further herein.

The process 100 may also include defining 120 a plurality of catheter profiles (e.g., providing a plurality of catheters, establishing a plurality of available catheter profiles, etc.). In some embodiments, the plurality of catheter profiles may draw from all different types of existing catheter profiles that are manufactured. In other embodiments, the plurality of catheter profiles may be a subset of catheter profiles (e.g., the catheter profiles that are presently available to a user). Further, the catheter profile may include a cardiac or neurostimulation lead delivery catheter (e.g., an electric or pacing lead), a peripheral catheter (e.g., balloon or stent delivery systems), a miniaturized leadless pacemaker delivery catheter, a catheter for advancing a guidewire, and/or a delivery catheter (e.g., to deliver drugs or medication).

Each catheter of the plurality of catheter profiles may include a plurality of catheter parameters. For example, each of the catheter profiles may differ based on a variety of different parameters or characteristics. Specifically, the plurality of catheter parameters of the plurality of catheter profiles may define shapes (e.g., arclengths, curvatures), stiffness, compliance, lengths (e.g., of various stiffness or compliance), wall thickness, diameter, etc.

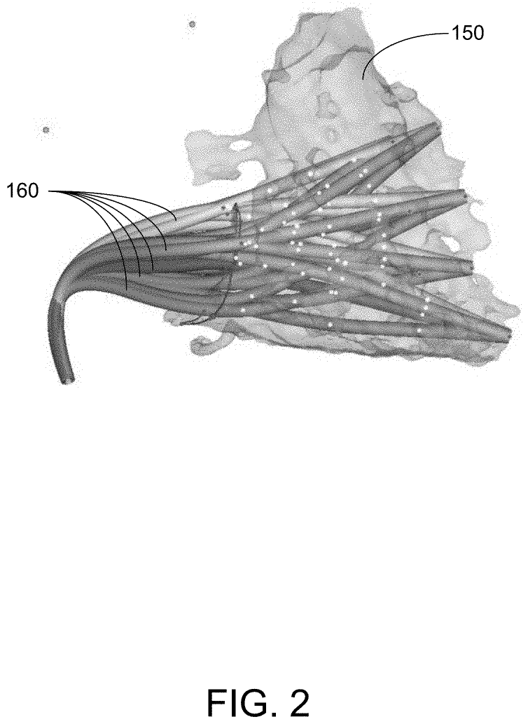

Also, the process 100 may include generating 130 a catheter path for each of the plurality of catheter profiles within the one or more anatomies. As shown in FIG. 2, generating the catheter path for each of the plurality of catheter profiles may include simulating the anatomy 150 and the catheter path 160 for each of the catheter profiles extending therethrough. For example, the anatomy 150 may be segmented based on portions that interact with the catheter profile and specific catheter anatomy contact points and implant targets may be measured. Further, the pre-shaped catheter paths 160 for each of the plurality of catheter profiles may be imported into the segmented anatomy, e.g., as shown in FIG. 2. These catheter paths may represent a variety of different catheter parameters or pre-shapes (e.g., rigid or compliant catheter sections). The catheter paths for each catheter profile may enforce shape and stiffness restrictions to ensure compatibility with anatomy. Thereafter, catheter paths that collide with the anatomy may be eliminated or considered incompatible, leaving the catheter paths that are possible. In one or more embodiments, the process may be repeated with multiple anatomies such that catheter paths may be generated for each anatomy of multiple anatomies.

Additionally, the process 100 may include determining 140 one or more optimal catheter profiles from a plurality of catheter profiles, as shown in FIG. 1. It is noted that the term “optimal” may refer to one or more catheter profiles that are best suited for a particular procedure or application or better suited than the remaining catheter profiles. Further, for example, optimal may be defined by a clinical outcome, which may be determined by the catheter profile's ability to deliver therapy in the correct location and in the correct trajectory. Further yet, for example, a good metric for catheter efficacy or determining an optimal catheter profile may be defined by procedure time (e.g., shorter procedure times may equal less radiation, less opportunity for infection, and/or more efficient operation room scheduling). Thereafter, in one or more embodiments, the optimized catheter profiles or shapes (e.g., including arclengths and curvatures) may be reported or displayed. In one or more embodiments, the optimized catheter profiles may be reported or displayed by, for example, computer display, IP sheet or matrix, using parameters to drive the user (e.g., the selector) into certain catheter profiles. In some embodiments, a computer decision tree may assist in potential catheter profile matches based on what you want to do. Ultimately, the various catheter profiles may be auto mapped for a specific anatomy based on success rate or performance.

In one or more embodiments, the process may include digital manipulation of a catheter profile of the plurality of catheter profiles in a computer heart model based on geometric boundary conditions (e.g., the plurality of anatomy parameters). For example, the orientation of the superior vena cava (SVC), location of the coronary sinus (CS) ostium, and angle of the CS body to the right atrium may assist in guiding the user through a series of virtual procedure steps aimed at getting and engaging the catheter profile with the CS. Once the location of the catheter tip is as close to the CS ostium as can be based on the geometric constraints of the anatomy and baseline shape of the catheter profile, the alignment of the catheter tip to the body of the CS may be the final indicator of how well or poorly the catheter profile may align with and engage the CS. Catheter profile designs that are closer to the CS ostium (e.g., across the patient population) and aligned well with the CS body after manipulation (e.g., across the patient population) may be deemed to be the best design (e.g., optimal) for that population

In one or more embodiments, the one or more optimal catheter profiles may include a single catheter profile that best suits the one or more anatomies. In other embodiments, the one or more optimal catheter profiles may include multiple catheter profiles that equally suit the one or more anatomies (e.g., essentially equivalents). For example, the one or more optimal catheter profiles may pass a specified threshold upon which the catheter profile is determined to be “optimal.” The different types of threshold may include proximity to target or implant locations, the orientation of the catheter tip relative to a target, fit within the anatomy of interest (e.g., knowing whether the catheter profile is too big or too small for its intended purpose before the catheter is inserted into the patient), etc. In one or more embodiments, the one or more optimal catheter profiles may include at least one of the one or more optimal catheter profiles that may be ranked or grouped into tiers. Further, the one or more optimal catheter profiles may be identified with various advantages or disadvantages relative to one another.

Depending on the application, generating a catheter path and determining one or more optimal catheter profiles may take several forms. For example, in one or more embodiments, determining the one or more optimal catheter profiles may include eliminating that catheter profiles that are not applicable to or compatible with the one or more anatomies (e.g., leaving the catheter profiles that perform the intended function). In other words, the catheter profiles of the plurality of catheter profiles for which the catheter path collides with portions of the one or more anatomy may be eliminated. As such, the one or more optimal catheter profiles may simply be a collection of catheter profiles that were not eliminated from the plurality of catheter profiles (e.g., ones that are compatible).

In one or more embodiments, determining the one or more optimal catheter profiles from the plurality of catheter profiles may include determining the one or more optimal catheter profiles for a demographic group (e.g., from a plurality of demographic groups). For example, the demographic group may be strongly associated with one or more anatomy parameters that specifically fit one or more catheter profiles of the plurality of catheter profiles. In other words, the one or more optimal catheter profiles may be identified for a specific demographic group.

In some embodiments, it may be valuable for the catheter profile to interact with different portions of the anatomy. For example, determining the one or more optimal catheter profiles from the plurality of catheter profiles may include identifying locations within the one or more anatomies (e.g., based on the plurality of anatomy parameters) and comparing the plurality of catheter profiles to desired locations within the one or more anatomies. Specifically, in one or more embodiments, the catheter profile may contact the right ventricular outflow tract in a direction perpendicular to the septal wall in order to deliver a (e.g., miniaturized) leadless pacemaker, or the catheter profile may contact the CS ostium in a direction allowing cannulation in order to deliver a left heart lead, or the catheter profile may contact the atrial septum in order to puncture into the bundle of His, High or Mid septal wall (e.g., for LBBAP), etc. Also, in one or more embodiments, the catheter profile may extend within the pulmonary artery branch or branches to deliver a device/sensor into the branch or branches, or the catheter profile may extend within the lung airways (e.g., bronchioles) to excise tissue/biopsy, or the catheter profile may extend through coronary arteries for stent delivery, or the catheter profile may extend through the pulmonary veins for pulmonary vein isolation (e.g., ablation) and for cryo-therapy.

Further, in one or more embodiments, the identified locations may be used in Mitral valve repair (e.g., where catheters may be used to cross an existing valve or navigate through an existing valve frame) for structure heart cases. In one or more embodiments, for example, the catheter profile may travel along a contralateral approach in peripheral stenting of the legs (e.g., approach is in one leg, goes up and over through the abdomen, and back down the other leg). Therefore, in one or more embodiments, the process may include evaluating a position of each catheter profile within the anatomy (e.g., to determine whether the catheter profile may properly interact with a portion of the anatomy as desired).

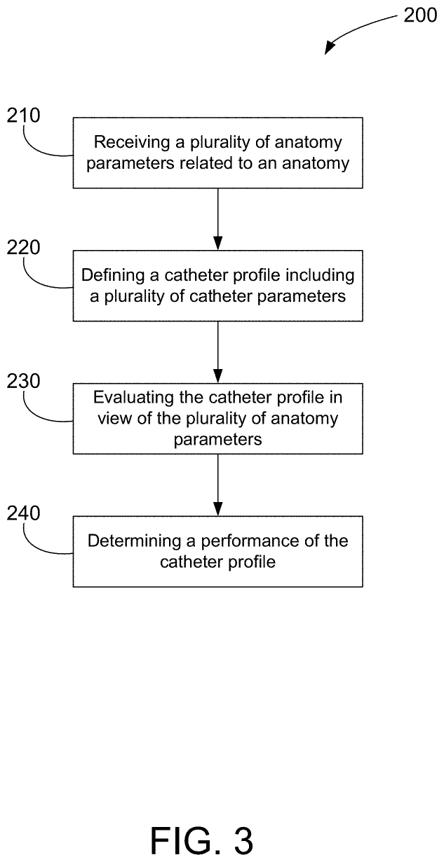

FIG. 3 illustrates a method 200 of evaluating a catheter profile in view of an anatomy having a plurality of anatomy parameters. For example, in one or more embodiments, a user may only have access to a single catheter profile having a set of parameters or characteristics. As described herein, the catheter profile may be evaluated (e.g., how well does the catheter profile work) as it pertains to the efficacy (e.g., for implantation, stimulation, therapy (e.g., drug, biologic, or electrical) delivery, fixation, etc.) for a specific anatomy.

The method 200 may include receiving 210 a plurality of anatomy parameters related to an anatomy. In other words, specific parameters or characteristics of a patient's anatomy may be input into a tool (e.g., through a system or controller) in order to evaluate the catheter profile. The plurality of anatomy parameters may include venous or arterial boundary shapes and distances, locations of anatomic targets relative to other anatomic landmarks, atrial wall locations, valve structure, coronary sinus ostia, coronary artery takeoffs, ventricular free and septal wall locations, orientation, endocardial shape (e.g., concave, convex), etc.

The plurality of anatomy parameters may be derived in a variety of different ways. For example, the one or more anatomies may be imaged to determine the plurality of anatomy parameters. Specifically, the imaging may include magnetic resonance imaging (MRI), fluoroscopy, cineradiography, biplane fluoroscopy, biplane cineradiography, computed tomography (CT), intraoperative 2D/3D imaging system (O-Arm), ultrasound (US), a transesophageal echocardiography (TEE), intra-cardiac echocardiography (ICE), transthoracic echocardiography (TTE), intravascular ultrasound (IVUS), electromechanical wave imaging (EWI), neuro-endoscopy, single photon emission computed tomography (SPECT), magnetic resonance angiography (MRA), computed tomography angiography (CTA), positron emission tomography (PET), optical coherence tomography (OCT), optical imaging spectroscopy (OIS), a magnetic resonance spectroscopy (MRS), dynamic susceptibility contrast (DSC) MRI, a fluid-attenuated inversion recovery (FLAIR), rotational angiography, infrared (IR) imaging, ultraviolet (UV) imaging, and/or the like. Also, the one or more anatomies may be measured in any suitable way such as, e.g., by segmenting anatomic boundaries from volumetric images (e.g., to extract anatomic boundaries) or by computing a best-fit volume from two or more perspective images of the anatomy. In one or more embodiments, 2D images (e.g., ultrasound planes) of the one or more anatomies may be measured by identifying (e.g., either manually or automatically) landmarks important to the performance of the catheter profile. Further, a user may manually input the plurality of anatomy parameters into a system or controller of the tool (e.g., due to known values of the plurality of anatomy parameters).

The method 200 may also include defining 220 a catheter profile having a plurality of catheter parameter profiles (e.g., providing a catheter, establishing a catheter profile, etc.). For example, the plurality of catheter parameters of the catheter profile may include shapes (e.g., arclengths, curvatures), stiffness, compliance, lengths (e.g., of various stiffness or compliance), wall thickness, diameter, etc. Further, the catheter profile may include a cardiac or neurostimulation lead delivery catheter (e.g., an electric or pacing lead), a peripheral catheter (e.g., balloon or stent delivery systems), a miniaturized leadless pacemaker delivery catheter, a catheter for advancing a guidewire, and/or a delivery catheter (e.g., to deliver drugs or medication).

Also, the method 200 may include evaluating 230 the catheter profile in view of the plurality of anatomy parameters. The catheter profile may be evaluated in view of the plurality of anatomy parameters in a variety of different ways. For example, evaluating the catheter profile by determining a position of the catheter profile relative to an implant and/or stimulation region. The plurality of anatomy parameters may be used to establish the location of the implant/stimulation region. In one or more embodiments, a particular portion of the catheter profile (e.g., a pacing lead, balloon, delivery port, etc.) may be evaluated to position the portion of the catheter profile relative to a location within the anatomy. For example, a location within the anatomy may be identified based on the plurality of anatomy parameters and the catheter profile may be compared to the locations within the anatomy. Also, for example, the catheter profile may be evaluated on whether or not the catheter profile is compatible with the anatomy.

In one or more embodiments, a demographic group may define the plurality of anatomy parameters. Therefore, a specific catheter profile may be evaluated in view of the demographic group representing the plurality of anatomy parameters. The demographic group may be identified within a variety of different categories. For example, the demographic group may be identified within one or more of height, BMI, ethnicity, gender, disease state, etc.

Additionally, the method 200 may include determining 240 a performance of the catheter profile. The performance of the catheter profile may be represented in a variety of different ways. For example, if evaluating the catheter profile by determining whether or not a catheter profile is compatible with the anatomy, the performance metric may be determined that the catheter profile is compatible or incompatible. Further, in one or more embodiments, the performance metric may be relayed to the physician by indicating the catheter profile is compatible or incompatible, by superimposing the catheter profile on the patient's anatomy and color-coding regions of the catheter profile that fall outside of or near the boundaries of the anatomy, by providing a list of catheter shapes that are compatible with the patient's anatomy, etc.

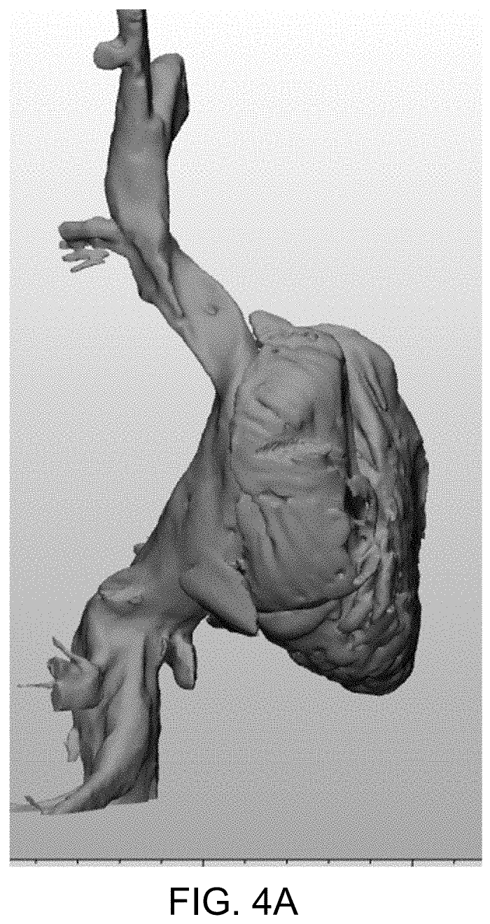

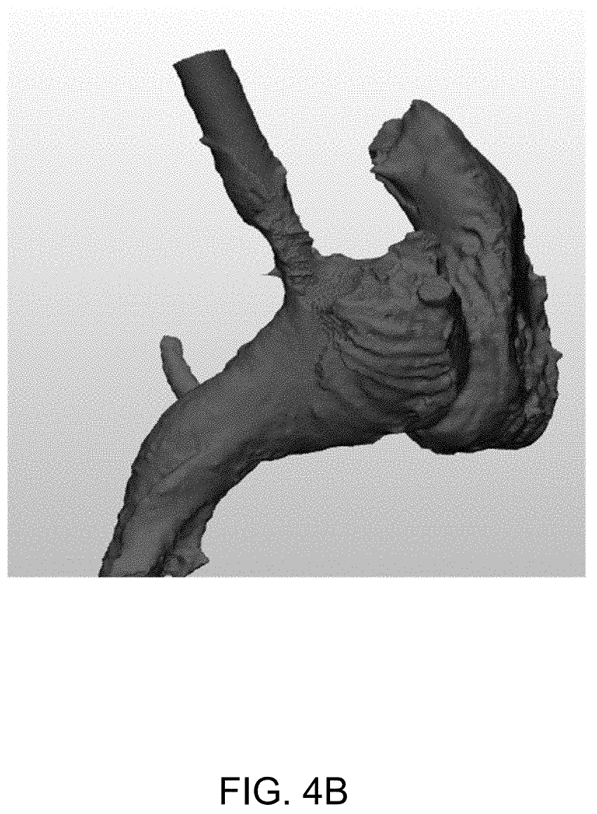

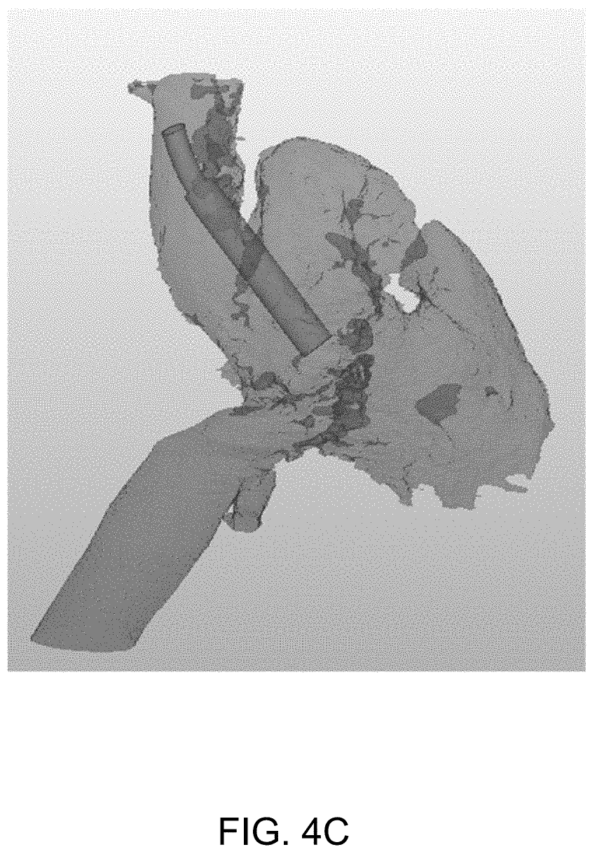

Also, for example, it is noted the potential great variation from anatomy to anatomy such that the performance of one catheter profile in one anatomy may not match the performance of the same catheter profile in another anatomy. For example, FIGS. 4A and 4B illustrate anatomic variation in the atrial superior vena cava (SVC)/inferior vena cava (IVC) junction between two different hearts. As such, the catheter profile may not be compatible with the heart illustrated in FIG. 4A and the catheter profile may be compatible with the heart illustrated in FIG. 4B. Further, for example, FIG. 4C illustrates a tool design (e.g., a catheter profile) for a human implant that does not fit the anatomy structure of an animal. This information allows the user to identify early on the expected performance of a given tool or catheter profile prior to further implementation or use.

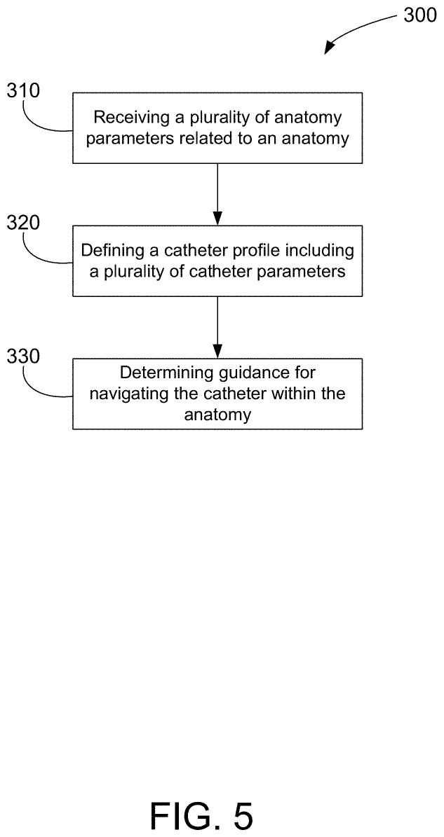

FIG. 5 illustrates a method 300 of determining guidance for navigating a catheter profile within an anatomy. For example, it may be beneficial for a user to understand the best path for navigating a specific catheter profile within a particular anatomy. By customizing the navigation based on the specifics for an individualized procedure, the user may more effectively carry out the procedure.

The method 300 may include receiving 310 a plurality of anatomy parameters related to an anatomy. In other words, specific parameters or characteristics of a patient's anatomy may be input into a tool (e.g., through a system or controller) in order to provide guidance for the catheter profile. The plurality of anatomy parameters may include venous or arterial boundary shapes and distances, locations of anatomic targets relative to other anatomic landmarks, atrial wall locations, valve structure, coronary sinus ostia, coronary artery takeoffs, ventricular free and septal wall locations, orientation, endocardial shape (e.g., concave, convex), etc.

The plurality of anatomy parameters may be derived in a variety of different ways. For example, the one or more anatomies may be imaged to determine the plurality of anatomy parameters. Specifically, the imaging may include magnetic resonance imaging (MRI), fluoroscopy, cineradiography, biplane fluoroscopy, biplane cineradiography, computed tomography (CT), intraoperative 2D/3D imaging system (O-Arm), ultrasound (US), a transesophageal echocardiography (TEE), intra-cardiac echocardiography (ICE), transthoracic echocardiography (TTE), intravascular ultrasound (IVUS), electromechanical wave imaging (EWI), neuro-endoscopy, single photon emission computed tomography (SPECT), magnetic resonance angiography (MRA), computed tomography angiography (CTA), positron emission tomography (PET), optical coherence tomography (OCT), optical imaging spectroscopy (OIS), a magnetic resonance spectroscopy (MRS), dynamic susceptibility contrast (DSC) MRI, a fluid-attenuated inversion recovery (FLAIR), rotational angiography, infrared (IR) imaging, ultraviolet (UV) imaging, and/or the like. Also, the one or more anatomies may be measured in any suitable way such as, e.g., by segmenting anatomic boundaries from volumetric images (e.g., to extract anatomic boundaries) or by computing a best-fit volume from two or more perspective images of the anatomy. In one or more embodiments, 2D images (e.g., ultrasound planes) of the one or more anatomies may be measured by identifying (e.g., either manually or automatically) landmarks important to the performance of the catheter profile. Further, a user may manually input the plurality of anatomy parameters into a system or controller of the tool (e.g., due to known values of the plurality of anatomy parameters).

The method 300 may also include defining 320 a catheter profile having a plurality of catheter parameters (e.g., providing a catheter, establishing a catheter profile, etc.). For example, the plurality of catheter parameters of the catheter profile may include shapes (e.g., arclengths, curvatures), stiffness, compliance, lengths (e.g., of various stiffness or compliance), wall thickness, diameter, etc. Further, the catheter profile may include a cardiac or neurostimulation lead delivery catheter (e.g., an electric or pacing lead), a peripheral catheter (e.g., balloon or stent delivery systems), a miniaturized leadless pacemaker delivery catheter, a catheter for advancing a guidewire, and/or a delivery catheter (e.g., to deliver drugs or medication).

Also, the method 300 may include determining 330 guidance for navigating the catheter profile within the anatomy. In one or more embodiments, the guidance for navigating the catheter profile within the anatomy may include an optimized path within the anatomy. In one or more embodiments, the guidance may include evaluating a position of the catheter profile within the anatomy. For example, the guidance may include displaying an image of the catheter profile extending along an optimized path within the anatomy. The imaging may be in any suitable form including, e.g., magnetic resonance imaging (MRI), fluoroscopy, cineradiography, biplane fluoroscopy, biplane cineradiography, computed tomography (CT), intraoperative 2D/3D imaging system (O-Arm), ultrasound (US), a transesophageal echocardiography (TEE), intra-cardiac echocardiography (ICE), transthoracic echocardiography (TTE), intravascular ultrasound (IVUS), electromechanical wave imaging (EWI), neuro-endoscopy, single photon emission computed tomography (SPECT), magnetic resonance angiography (MRA), computed tomography angiography (CTA), positron emission tomography (PET), optical coherence tomography (OCT), optical imaging spectroscopy (OIS), a magnetic resonance spectroscopy (MRS), dynamic susceptibility contrast (DSC) MRI, a fluid-attenuated inversion recovery (FLAIR), rotational angiography, infrared (IR) imaging, ultraviolet (UV) imaging, and/or the like.

In one or more embodiments, the system may also include determining an optimal site into which the catheter profile may be introduced into the patient. For example, the plurality of catheter parameters and the plurality of anatomy parameters may be considered to determine which introduction site may be most efficient for a given procedure.

Further, in one or more embodiments, the location of the catheter profile may be superposed on top of a previously recorded heart image. The physician may then overlay that image on a real time imaging and “follow the lead” to the correct anatomic location.

In one or more embodiments, a demographic group may define the plurality of anatomy parameters. Therefore, guidance for a specific catheter profile may be output for inserting the catheter profile within the anatomy based on the demographic group. The demographic group may be identified within a variety of different categories. For example, the demographic group may be identified within one or more of height, BMI, ethnicity, gender, disease state, etc.

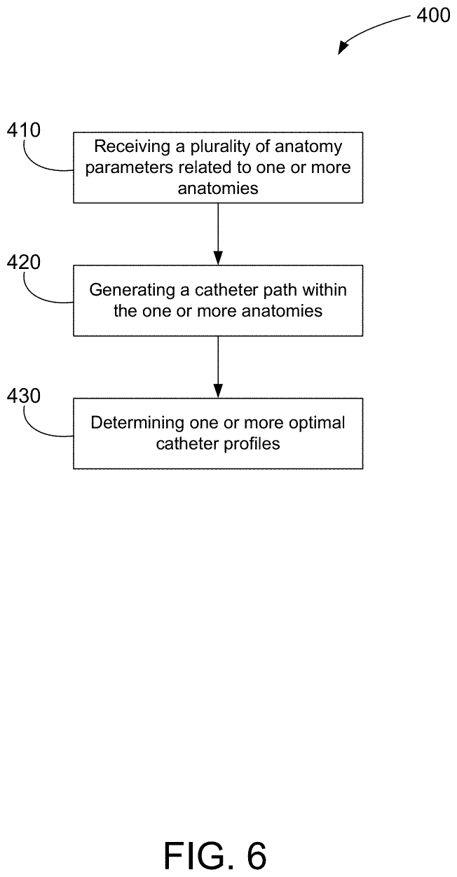

FIG. 6 illustrates a method 400 of determining or designing one or more optimal catheter profiles having a plurality of catheter parameters. For example, a catheter profile may be designed and manufactured to a particular specification of one person or multiple people (e.g., based on a plurality of anatomy parameters). In other words, in one or more embodiments, an optimal catheter profile can be designed according to a plurality of catheter parameters that is customized to that one person or multiple people. Further, in some embodiments, the optimal catheter profile may be manufactured in a short period of time (e.g., using additive manufacturing or 3D printing) to that set of catheter parameters. As such, a catheter profile could be designed and manufactured “on-demand” for that one person or multiple people. This may allow the user to design and manufacture an “optimal” catheter profile in a short amount of time such that the “optimal” catheter profile can be used almost immediately.

The method 400 may include receiving 410 a plurality of anatomy parameters related to one or more anatomies. In other words, specific parameters or characteristics of a patient's anatomy may be input into a tool (e.g., through a system or controller) in order to design the optimal catheter profile. The plurality of anatomy parameters may include venous or arterial boundary shapes and distances, locations of anatomic targets relative to other anatomic landmarks, atrial wall locations, valve structure, coronary sinus ostia, coronary artery takeoffs, ventricular free and septal wall locations, orientation, endocardial shape (e.g., concave, convex), etc.

The plurality of anatomy parameters may be derived in a variety of different ways. For example, the one or more anatomies may be imaged to determine the plurality of anatomy parameters. Specifically, the imaging may include magnetic resonance imaging (MRI), fluoroscopy, cineradiography, biplane fluoroscopy, biplane cineradiography, computed tomography (CT), intraoperative 2D/3D imaging system (O-Arm), ultrasound (US), a transesophageal echocardiography (TEE), intra-cardiac echocardiography (ICE), transthoracic echocardiography (TTE), intravascular ultrasound (IVUS), electromechanical wave imaging (EWI), neuro-endoscopy, single photon emission computed tomography (SPECT), magnetic resonance angiography (MRA), computed tomography angiography (CTA), positron emission tomography (PET), optical coherence tomography (OCT), optical imaging spectroscopy (OIS), a magnetic resonance spectroscopy (MRS), dynamic susceptibility contrast (DSC) MRI, a fluid-attenuated inversion recovery (FLAIR), rotational angiography, infrared (IR) imaging, ultraviolet (UV) imaging, and/or the like. Also, the one or more anatomies may be measured in any suitable way such as, e.g., by segmenting anatomic boundaries from volumetric images (e.g., to extract anatomic boundaries) or by computing a best-fit volume from two or more perspective images of the anatomy. In one or more embodiments, 2D images (e.g., ultrasound planes) of the one or more anatomies may be measured by identifying (e.g., either manually or automatically) landmarks important to the performance of the catheter profile. Further, a user may manually input the plurality of anatomy parameters into a tool (e.g., using a system or controller).

The method 400 may also include generating 420 a catheter path within the one or more anatomies. The catheter path may take into account the plurality of anatomy parameters to determine the optimal path for a specific application. In one or more embodiments, the catheter path may include points of interest (e.g., implant and/or stimulation points) within the one or more anatomies through which the catheter path should travel.

Also, the method 400 may include designing 430 one or more optimal catheter profiles according to the catheter path. Each optimal catheter profile having a plurality of catheter parameters. Based on the catheter path, the one or more optimal catheter profiles may be designed according to the corresponding plurality of catheter parameters. As such, the one or more optimal catheter profiles may include features at specific locations (e.g., anchors, leads, balloons, deliver ports, lumens, markers, etc.) to interact with the points of interest determined along the catheter path.

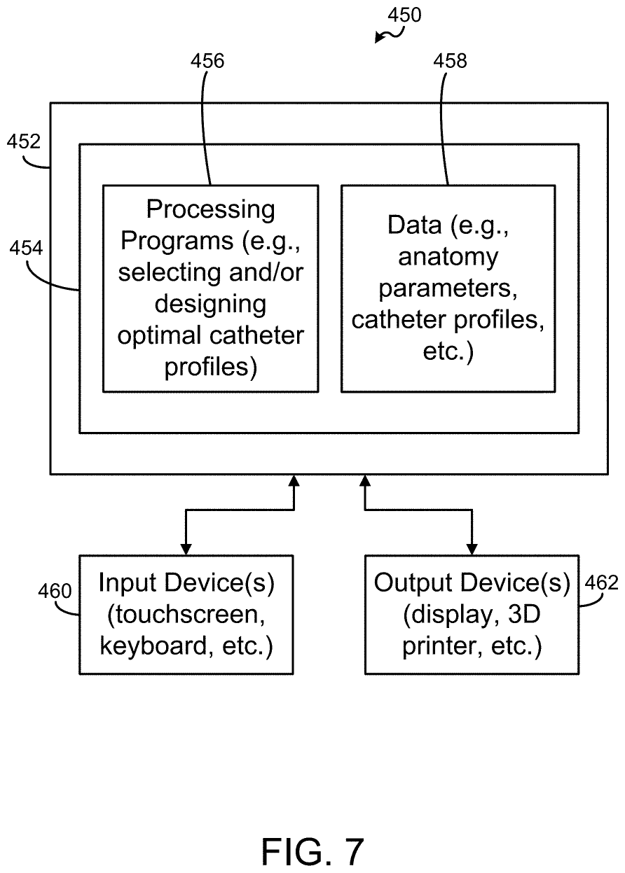

A functional block diagram of an exemplary system 450 for use with catheter design tools and methods thereof for selecting or designing an optimal catheter profile for a particular patient or group of patients, as described herein, is illustrated in FIG. 7. The system 450 may be representative of any systems or devices described herein for use in determining one or more optimal catheter profiles from a plurality of catheter profiles based on a plurality of catheter parameters of the plurality of catheter profiles and a plurality of anatomy parameters as described in combination with FIG. 1, for use in evaluating a catheter profile in view of an anatomy having a plurality of anatomy parameters as described in combination with FIG. 3, for use in determining guidance for navigating a catheter profile within an anatomy as described in combination with FIG. 5, and/or for use in determining one or more optimal catheter profiles having a plurality of catheter parameters as described in combination with FIG. 6.

The system 450 may include a processing apparatus, or a processor, 452, input device(s) 460, and output device(s) 462. Generally, the input device 460 may be operably coupled to the processing apparatus 452 and may include any one or more devices configured to allow a user to provide input to the processing apparatus 452. The input device 460 may include any apparatus, structure, or devices configured to receive input from a user. For example, the input device 460 may include one or more keyboards, mice, microphones, touchscreens, smart pens, etc.

Additionally, the input device 460 may be further described in terms of the various input modalities. For example, the input device 460 may be configured to receive touch inputs from a user using a touchscreen display, keystrokes of a keyboard, voice commands, gesture commands, etc. In essence, the input device 460 may be configured to provide input capabilities in various locations, mediums, and conditions to, e.g., receive a plurality of anatomy parameters, a plurality of catheter profiles, a plurality of catheter parameters, etc. In one or more embodiments, the processing apparatus 452 may be configured to receive measurements from the various imaging modalities described herein.

The system 450 may additionally include output device(s) 462 operably coupled to the processing apparatus 452. The output device(s) 462 may include any one or more devices configured to provide visual and tactile information to a user. For example, the output device(s) 462 may include one or more monitors, screens, liquid crystal display panels, organic light emitting diode panels, tactile displays (e.g., braille or moveable bump displays), lights, etc. Also, for example, the output device(s) 462 may include a decision matrix or tree to follow based on specific parameters under the current circumstances. Further, in one or more embodiments, the output device(s) 462 may include a 3D printer or additive manufacturing device to create or manufacture a catheter profile on location (e.g., in real time).

Further, the processing apparatus 452 includes data storage 454. Data storage 454 allows for access to processing programs or routines 456 and one or more other types of data 458 that may be employed to carry out the exemplary methods, processes, and algorithms of selecting and/or designing an optimal catheter profile for a particular patient or group of patients. For example, processing programs or routines 456 may include programs or routines for performing computational mathematics, matrix mathematics, Fourier transforms, image registration processes, compression algorithms, calibration algorithms, image construction algorithms, inversion algorithms, signal processing algorithms, normalizing algorithms, deconvolution algorithms, averaging algorithms, standardization algorithms, comparison algorithms, vector mathematics, location tracking, audio comparison, learning algorithms, biomimetic computation, or any other processing required to implement one or more embodiments as described herein.

Data 458 may include, for example, anatomy parameters, catheter profiles, catheter parameters, or any other data that may be necessary for carrying out the one or more processes or methods described herein.

In one or more embodiments, the system 450 may be controlled using one or more computer programs executed on programmable computers, such as computers that include, for example, processing capabilities (e.g., microcontrollers, programmable logic devices, etc.), data storage (e.g., volatile or non-volatile memory and/or storage elements), input devices, and output devices. Program code and/or logic described herein may be applied to input data to perform functionality described herein and generate desired output information. The output information may be applied as input to one or more other devices and/or processes as described herein or as would be applied in a known fashion.

The programs used to implement the processes described herein may be provided using any programmable language, e.g., a high-level procedural and/or object orientated programming language that is suitable for communicating with a computer system. Any such programs may, for example, be stored on any suitable device, e.g., a storage media, readable by a general or special purpose program, computer or a processor apparatus for configuring and operating the computer when the suitable device is read for performing the procedures described herein. In other words, at least in one embodiment, the system 450 may be controlled using a computer readable storage medium, configured with a computer program, where the storage medium so configured causes the computer to operate in a specific and predefined manner to perform functions described herein.

The processing apparatus 452 may be, for example, any fixed or mobile computer system (e.g., a personal computer or minicomputer). The exact configuration of the computing apparatus is not limiting and essentially any device capable of providing suitable computing capabilities and control capabilities (e.g., control the display of the system 450, the acquisition of data,) may be used. Further, various peripheral devices, such as a computer display, mouse, keyboard, memory, printer, scanner, etc. are contemplated to be used in combination with the processing apparatus 452. Further, in one or more embodiments, the data 458 may be analyzed by a user, used by another machine that provides output based thereon, etc. As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by processing apparatus 452 described herein. Also, as described herein, a file in user-readable format may be any representation of data (e.g., ASCII text, binary numbers, hexadecimal numbers, decimal numbers, audio, graphical) presentable on any medium (e.g., paper, a display, sound waves, etc.) readable and/or understandable by a user.

In view of the above, it will be readily apparent that the functionality as described in one or more embodiments according to the present disclosure may be implemented in any manner as would be known to one skilled in the art. As such, the computer language, the computer system, or any other software/hardware that is to be used to implement the processes described herein shall not be limiting on the scope of the systems, processes or programs (e.g., the functionality provided by such systems, processes or programs) described herein.

The methods described in this disclosure, including those attributed to the systems, or various constituent components, may be implemented, at least in part, in hardware, software, firmware, or any combination thereof. For example, various aspects of the techniques may be implemented by the processing apparatus, which may use one or more processors such as, e.g., one or more microprocessors, DSPs, ASICs, FPGAs, CPLDs, microcontrollers, or any other equivalent integrated or discrete logic circuitry, as well as any combinations of such components, image processing devices, or other devices. The term “processing apparatus,” “processor,” or “processing circuitry” may generally refer to any of the foregoing logic circuitry, alone or in combination with other logic circuitry, or any other equivalent circuitry. Additionally, the use of the word “processor” may not be limited to the use of a single processor but is intended to connote that at least one processor may be used to perform the exemplary methods and processes described herein.

Such hardware, software, and/or firmware may be implemented within the same device or within separate devices to support the various operations and functions described in this disclosure. In addition, any of the described components may be implemented together or separately as discrete but interoperable logic devices. Depiction of different features, e.g., using block diagrams, etc., is intended to highlight different functional aspects and does not necessarily imply that such features must be realized by separate hardware or software components. Rather, functionality may be performed by separate hardware or software components, or integrated within common or separate hardware or software components.

When implemented in software, the functionality ascribed to the systems, devices and methods described in this disclosure may be embodied as instructions on a computer-readable medium such as RAM, ROM, NVRAM, EEPROM, FLASH memory, magnetic data storage media, optical data storage media, or the like. The instructions may be executed by the processing apparatus to support one or more aspects of the functionality described in this disclosure.

Thus, various embodiments described herein are disclosed. It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

All references and publications cited herein are expressly incorporated herein by reference in their entirety for all purposes, except to the extent any aspect directly contradicts this disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

As used herein, the term “configured to” may be used interchangeably with the terms “adapted to” or “structured to” unless the content of this disclosure clearly dictates otherwise.

Th singular forms “a,” “an,” and “the” encompass embodiments having plural referents unless its context clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

### ILLUSTRATIVE EMBODIMENTS

While the present disclosure is not so limited, an appreciation of various aspects of the disclosure will be gained through a discussion of the specific examples and illustrative embodiments provided below. Various modifications of the examples and illustrative embodiments, as well as additional embodiments of the disclosure, will become apparent herein.

A1. A method comprising:
receiving a plurality of anatomy parameters related to one or more anatomies;
defining a plurality of catheter profiles, wherein each catheter profile of the plurality of catheter profiles comprises a plurality of catheter parameters;
generating a catheter path for each of the plurality of catheter profiles within the one or more anatomies; and
determining one or more optimal catheter profiles from the plurality of catheter profiles.
   A2. The method according to any A embodiment, wherein determining the one or more optimal catheter profiles from the plurality of catheter profiles comprises:
eliminating catheter profiles of the plurality catheter profiles for which the catheter path collides with portions of the one or more anatomies resulting in a subset of the plurality of catheter profiles; and
determining the one or more optimal catheter profiles from the subset of the plurality of catheter profiles.
   A3. The method according to any A embodiment, further comprising imaging the one or more anatomies to determine the plurality of anatomy parameters.
   A4. The method according to any A embodiment, wherein the one or more anatomies correlate to a demographic group and wherein determining one or more optimal catheter profiles from the plurality of catheter profiles comprises determining the one or more optimal catheter profiles for the demographic group.
   A5. The method according to embodiment A4, wherein the demographic group is identified within one or more of height, BMI, ethnicity, gender, disease state, etc.
   A6. The method according to any A embodiment, wherein the plurality of catheter parameters of the plurality of catheter profiles define shapes (e.g., arclengths, curvatures, etc.), stiffness, compliance, lengths, etc.
   A7. The method according to any A embodiment, wherein determining the one or more optimal catheter profiles from the plurality of catheter profiles comprises identifying locations within the one or more anatomies based on the plurality of anatomy parameters and comparing the plurality of catheter profiles to the locations within the one or more anatomies.
   A8. The method according to any A embodiment, further comprising ranking at least one of the one or more optimal catheter profiles.
   A9. The method according to any A embodiment, further comprising grouping the one or more anatomies into a plurality of demographic groups, and wherein determining one or more optimal catheter profiles from the plurality of catheter profiles comprises determining the one or more optimal catheter profiles for a demographic group of the plurality of demographic groups.
   A10. The method according to any A embodiment, wherein the catheter profile comprises a lead catheter or a drug delivery catheter.
   B1. A system comprising:
a storage apparatus storing a plurality of catheter profiles, wherein each catheter profile of the plurality of catheter profiles comprises a plurality of catheter parameters; and
computing apparatus comprising processing circuitry and operably coupled to the storage apparatus, the computing apparatus configured to:
   receive a plurality of anatomy parameters related to one or more anatomies;
   determine a catheter path for each of the plurality of catheter profiles within the one or more anatomies; and
   select one or more optimal catheter profiles from the plurality of catheter profiles.
      B2. The system according to any B embodiment, wherein selecting the one or more optimal catheter profiles from the plurality of catheter profiles comprises:
eliminating catheter profiles of the plurality catheter profiles for which the catheter path collides with portions of the one or more anatomies resulting in a subset of the plurality of catheter profiles; and
determining the one or more optimal catheter profiles from the subset of the plurality of catheter profiles.
   B3. The system according to any B embodiment, wherein the computing apparatus is further configured to image the one or more anatomies to determine the plurality of anatomy parameters.
   B4. The system according to any B embodiment, wherein the one or more anatomies correlate to a demographic group and wherein selecting one or more optimal catheter profiles from the plurality of catheter profiles comprises determining the one or more optimal catheter profiles for the demographic group.
   B5. The system according to embodiment B4, wherein the demographic group is identified within one or more of height, BMI, ethnicity, gender, disease state, etc.
   B6. The system according to any B embodiment, wherein the plurality of catheter parameters of the plurality of catheter profiles define shapes (e.g., arclengths, curvatures, etc.), stiffness, compliance, lengths, etc.
   B7. The system according to any B embodiment, wherein selecting the one or more optimal catheter profiles from the plurality of catheter profiles comprises identifying locations within the one or more anatomies based on the plurality of anatomy parameters and comparing the plurality of catheter profiles to the locations within the one or more anatomies.
   B8. The system according to any B embodiment, wherein the computing apparatus is further configured to rank at least one of the one or more optimal catheter profiles.
   B9. The system according to any B embodiment, wherein the computing apparatus is further configured to group the one or more anatomies into a plurality of demographic groups, and wherein selecting one or more optimal catheter profiles from the plurality of catheter profiles comprises determining the one or more optimal catheter profiles for a demographic group of the plurality of demographic groups.
   B10. The system according to any B embodiment, wherein the catheter profile comprises a lead catheter or a drug delivery catheter.
   C1. A method comprising:
receiving a plurality of anatomy parameters related to an anatomy;
defining a catheter profile comprising a plurality of catheter parameters;
evaluating the catheter profile in view of the plurality of anatomy parameters; and
determining a performance of the catheter profile.
   C2. The method according to any C embodiment, wherein evaluating the catheter profile in view of the plurality of anatomy parameters comprises determining a position of the catheter profile relative to an implant/stimulation region.
   C3. The method according to any C embodiment, wherein evaluating the catheter profile in view of the plurality of anatomy parameters comprises determining whether the catheter profile is incompatible with the anatomy.
   C4. The method according to any C embodiment, further comprising imaging the anatomy to determine the plurality of anatomy parameters.
   C5. The method according to any C embodiment, wherein a demographic group defines the plurality of anatomy parameters, wherein the catheter profile is evaluated in view of the demographic group.
   C6. The method according to embodiment C5, wherein the demographic group is identified within one or more of height, BMI, ethnicity, gender, disease state, etc.
   C7. The method according to any C embodiment, wherein the plurality of catheter parameters of the catheter profile defines shapes (e.g., arclengths, curvatures, etc.), stiffness, compliance, lengths, etc.
   C8. The method according to any C embodiment, wherein evaluating the catheter profile in view of the plurality of anatomy parameters comprises identifying locations within the anatomy based on the plurality of anatomy parameters and comparing the catheter profile to the locations within the anatomy.
   C9. The method according to any C embodiment, wherein the catheter profile comprises a lead catheter or a drug delivery catheter.
   D1. A system comprising:
a storage apparatus storing a catheter profile, wherein the catheter profile comprises a plurality of catheter parameters; and
computing apparatus comprising processing circuitry and operably coupled to the storage apparatus, the computing apparatus configured to:
   receive a plurality of anatomy parameters related to an anatomy;
   evaluate the catheter profile in view of the plurality of anatomy parameters; and
   determine a performance of the catheter profile.
      D2. The system according to any D embodiment, wherein evaluating the catheter profile in view of the plurality of anatomy parameters comprises determining a position of the catheter profile relative to an implant/stimulation region.
      D3. The system according to any D embodiment, wherein evaluating the catheter profile in view of the plurality of anatomy parameters comprises determining whether the catheter profile is incompatible with the anatomy.
      D4. The system according to any D embodiment, wherein the computing apparatus is further configured to image the anatomy to determine the plurality of anatomy parameters.
      D5. The system according to any D embodiment, wherein a demographic group defines the plurality of anatomy parameters, wherein the catheter profile is evaluated in view of the demographic group.
      D6. The system according to embodiment D5, wherein the demographic group is identified within one or more of height, BMI, ethnicity, gender, disease state, etc.
      D7. The system according to any D embodiment, wherein the plurality of catheter parameters of the catheter profile defines shapes (e.g., arclengths, curvatures, etc.), stiffness, compliance, lengths, etc.
      D8. The system according to any D embodiment, wherein evaluating the catheter profile in view of the plurality of anatomy parameters comprises identifying locations within the anatomy based on the plurality of anatomy parameters and comparing the catheter profile to the locations within the anatomy.
      D9. The system according to any D embodiment, wherein the catheter profile comprises a lead catheter or a drug delivery catheter.
      E1. A method comprising:
receiving a plurality of anatomy parameters related to an anatomy;
defining a catheter profile comprising a plurality of catheter parameters; and
determining guidance for navigating the catheter profile within the anatomy.
   E2. The method according to any E embodiment, wherein the guidance for navigating the catheter profile within the anatomy comprises an optimized path within the anatomy.
   E3. The method according to any E embodiment, wherein determining guidance for navigating the catheter profile within the anatomy comprises displaying an image of the catheter profile extending along an optimized path within the anatomy.
   E4. The method according to any E embodiment, further comprising imaging the anatomy to determine the plurality of anatomy parameters.
   E5. The method according to any E embodiment, wherein a demographic group defines the plurality of anatomy parameters, wherein the guidance determined for navigating the catheter profile within the anatomy is determined in view of the demographic group.
   E6. The method according to embodiment E5, wherein the demographic group is identified within one or more of height, BMI, ethnicity, gender, disease state, etc.
   E7. The method according to any E embodiment, wherein determining guidance for navigating the catheter profile within the anatomy comprises evaluating a position of the catheter profile relative to an implant/stimulation region.
   E8. The method according to any E embodiment, wherein the plurality of catheter parameters of the catheter profile defines shapes (e.g., arclengths, curvatures, etc.), stiffness, compliance, lengths, etc.
   E9. The method according to any E embodiment, wherein the catheter profile comprises a lead catheter or a drug delivery catheter.
   F1. A system comprising:
a storage apparatus storing a catheter profile, wherein the catheter profile comprises a plurality of catheter parameters; and
computing apparatus comprising processing circuitry and operably coupled to the storage apparatus, the computing apparatus configured to:
   receive a plurality of anatomy parameters related to an anatomy; and
   determine guidance for navigating the catheter profile within the anatomy.
      F2. The system according to any F embodiment, wherein the guidance for navigating the catheter profile within the anatomy comprises an optimized path within the anatomy.
      F3. The system according to any F embodiment, wherein determining guidance for navigating the catheter profile within the anatomy comprises displaying an image of the catheter profile extending along an optimized path within the anatomy.
      F4. The system according to any F embodiment, wherein the computing apparatus is further configured to image the anatomy to determine the plurality of anatomy parameters.
      F5. The system according to any F embodiment, wherein a demographic group defines the plurality of anatomy parameters, wherein the guidance determined for navigating the catheter profile within the anatomy is determined in view of the demographic group.
      F6. The system according to embodiment F5, wherein the demographic group is identified within one or more of height, BMI, ethnicity, gender, disease state, etc.
      F7. The system according to any F embodiment, wherein determining guidance for navigating the catheter profile within the anatomy comprises evaluating a position of the catheter profile relative to an implant/stimulation region.
      F8. The system according to any F embodiment, wherein the plurality of catheter parameters of the catheter profile defines shapes (e.g., arclengths, curvatures, etc.), stiffness, compliance, lengths, etc.
      F9. The system according to any F embodiment, wherein the catheter profile comprises a lead catheter or a drug delivery catheter.
      G1. A method comprising:
receiving a plurality of anatomy parameters related to one or more anatomies;
generating a catheter path within the one or more anatomies; and
determining one or more optimal catheter profiles according to the catheter path, each optimal catheter profile having a plurality of catheter parameters.
   G2. The method according to any G embodiment, further comprising imaging the one or more anatomies to determine the plurality of anatomy parameters.
   G3. The method according to any G embodiment, further comprising manufacturing the one or more optimal catheter profiles using 3D printing.
   G4. The method according to any G embodiment, wherein the plurality of catheter parameters of the one or more optimal catheter profiles define shapes (e.g., arclengths, curvatures, etc.), stiffness, compliance, lengths, etc.
   G5. The method according to any G embodiment, wherein generating a catheter path within the one or more anatomies comprises identifying locations within the one or more anatomies based on the plurality of anatomy parameters and comparing the catheter path to the locations identified within the one or more anatomies.
   H1. A system comprising:
a storage apparatus; and
computing apparatus comprising processing circuitry and operably coupled to the storage apparatus, the computing apparatus configured to:
   receive a plurality of anatomy parameters related to one or more anatomies;
   generate a catheter path within the one or more anatomies; and
   determine one or more optimal catheter profiles according to the catheter path, each optimal catheter profile having a plurality of catheter parameters.
      H2. The system according to any H embodiment, wherein the computing apparatus is further configured to image the one or more anatomies to determine the plurality of anatomy parameters.
      H3. The system according to any H embodiment, wherein the computing apparatus is further configured to manufacture the one or more optimal catheter profiles using 3D printing.
      H4. The system according to any H embodiment, wherein the plurality of catheter parameters of the one or more optimal catheter profiles define shapes (e.g., arclengths, curvatures, etc.), stiffness, compliance, lengths, etc.
      H5. The system according to any H embodiment, wherein generating a catheter path within the one or more anatomies comprises identifying locations within the one or more anatomies based on the plurality of anatomy parameters and comparing the catheter path to the locations identified within the one or more anatomies.

What is claimed:

1. A method comprising:
receiving a plurality of anatomy parameters related to one or more anatomies;
defining a plurality of catheter profiles, wherein each catheter profile of the plurality of catheter profiles comprises a plurality of catheter parameters;
generating a catheter path for each of the plurality of catheter profiles within the one or more anatomies; and
determining one or more optimal catheter profiles from the plurality of catheter profiles.

2. The method of claim 1, wherein determining the one or more optimal catheter profiles from the plurality of catheter profiles comprises:
eliminating catheter profiles of the plurality catheter profiles for which the catheter path collides with portions of the one or more anatomies resulting in a subset of the plurality of catheter profiles; and
determining the one or more optimal catheter profiles from the subset of the plurality of catheter profiles.

3. The method of claim 1, further comprising imaging the one or more anatomies to determine the plurality of anatomy parameters.

4. The method of claim 1, wherein the one or more anatomies correlate to a demographic group and wherein determining one or more optimal catheter profiles from the plurality of catheter profiles comprises determining the one or more optimal catheter profiles for the demographic group.

5. The method of claim 4, wherein the demographic group is identified within one or more of height, BMI, ethnicity, gender, and disease state.

6. The method of claim 1, wherein the plurality of catheter parameters of the plurality of catheter profiles define one or more of shapes, stiffness, compliance, lengths, wall thickness, and diameter.

7. The method of claim 1, wherein determining the one or more optimal catheter profiles from the plurality of catheter profiles comprises identifying locations within the one or more anatomies based on the plurality of anatomy parameters and comparing the plurality of catheter profiles to the locations within the one or more anatomies.

8. The method of claim 1, further comprising ranking at least one of the one or more optimal catheter profiles.

9. The method of claim 1, further comprising grouping the one or more anatomies into a plurality of demographic groups, and wherein determining one or more optimal catheter profiles from the plurality of catheter profiles comprises determining the one or more optimal catheter profiles for a demographic group of the plurality of demographic groups.

10. The method of claim 1, wherein the catheter profile comprises one or more of a cardiac or neurostimulation lead delivery catheter, a peripheral catheter, a miniaturized leadless pacemaker delivery catheter, a catheter for advancing a guidewire, and a drug delivery catheter.

11. A system comprising:
a storage apparatus storing a plurality of catheter profiles, wherein each catheter profile of the plurality of catheter profiles comprises a plurality of catheter parameters; and
computing apparatus comprising processing circuitry and operably coupled to the storage apparatus, the computing apparatus configured to:
receive a plurality of anatomy parameters related to one or more anatomies;
determine a catheter path for each of the plurality of catheter profiles within the one or more anatomies; and
select one or more optimal catheter profiles from the plurality of catheter profiles.

12. The system of claim 11, wherein selecting the one or more optimal catheter profiles from the plurality of catheter profiles comprises:
eliminating catheter profiles of the plurality catheter profiles for which the catheter path collides with portions of the one or more anatomies resulting in a subset of the plurality of catheter profiles; and
determining the one or more optimal catheter profiles from the subset of the plurality of catheter profiles.

13. The system of claim 11, wherein the computing apparatus is further configured to image the one or more anatomies to determine the plurality of anatomy parameters.

14. The system of claim 11, wherein the one or more anatomies correlate to a demographic group and wherein selecting one or more optimal catheter profiles from the plurality of catheter profiles comprises determining the one or more optimal catheter profiles for the demographic group.

15. The system of claim 14, wherein the demographic group is identified within one or more of height, BMI, ethnicity, gender, and disease state.

16. The system of claim 11, wherein the plurality of catheter parameters of the plurality of catheter profiles define one or more of shapes, stiffness, compliance, lengths, wall thickness, and diameter.

17. The system of claim 11, wherein selecting the one or more optimal catheter profiles from the plurality of catheter profiles comprises identifying locations within the one or more anatomies based on the plurality of anatomy parameters and comparing the plurality of catheter profiles to the locations within the one or more anatomies.

18. The system of claim 11, wherein the computing apparatus is further configured to rank at least one of the one or more optimal catheter profiles.

19. The system of claim 11, wherein the computing apparatus is further configured to group the one or more anatomies into a plurality of demographic groups, and wherein selecting one or more optimal catheter profiles from the plurality of catheter profiles comprises determining the one or more optimal catheter profiles for a demographic group of the plurality of demographic groups.

20. The system of claim 11, wherein the catheter profile comprises one or more of a cardiac or neurostimulation lead delivery catheter, a peripheral catheter, a miniaturized leadless pacemaker delivery catheter, a catheter for advancing a guidewire, and a drug delivery catheter.
